(19) United States
(12) Patent Application Publication
(10) Pub. No.: US 2011/0033376 A1
(43) Pub. Date: Feb. 10, 2011

(54) Treatment of Melanoma

(76) Inventors: Malcolm Philip Young, Tyne & Wear (GB); Catherine Mary Thomas, Newcastle Upon Tyne (GB); Olusola Clement Idowu, Tyne & Wear (GB); Julie Anne Charlton, Tyne & Wear (GB)

Correspondence Address: K&L Gates LLP, STATE STREET FINANCIAL CENTER, One Lincoln Street, BOSTON, MA, 02111-2950, US

(21) Appl. No.: 12/666,935

(22) PCT Filed: Jul. 4, 2008
(86) PCT No.: PCT/GB08/02320

§ 371 Date: Oct. 4, 2010

Related U.S. Application Data
(60) Provisional application No. 60/967,837, filed on Sep. 7, 2007.

(30) Foreign Application Priority Data
Jul. 6, 2007 (GB) 0713116.2

(51) Int. Cl. A61K 51/00; C07D 311/80; A61K 31/352; A61K 39/00; A61P 35/00; A61P 35/04

(52) U.S. Cl. 424/1.11; 549/390; 514/454; 424/277.1

(57) ABSTRACT

There is described dexanabinol, or a derivative thereof, for the treatment of melanoma. There is also described a method of treating a patient suffering from melanoma.

### FIELD OF THE INVENTION

The present invention provides medicaments and methods for the treatment of melanoma. More particularly the invention provides dexanabinol, or a derivative thereof, for the treatment of melanoma.

### BACKGROUND

Incidence of melanoma cases has doubled every year since the 1940s. Melanoma is now the sixth most common cancer in men, and the seventh most common cancer in women. Its incidence is increasing in all parts of the world (Parker, S et al, 1997). The 5 year survival rate for melanoma is 30 to 40%, with malignant melanoma carrying the highest risk of mortality from metastasis (Jemal et al, 2001); Spread of the disease to distant organs such as liver, bone and brain, reduces the 5 year survival to less than 12%. There is currently no effective long-term treatment for patients with metastatic (Stage IV) melanoma. Standard chemotherapy regimens do not impart a significant long term survival benefit in these patients, and chemotherapy may be associated with a degree of morbidity due to toxicity. There is an obvious need to develop new targeted therapies for melanoma, both to prevent cancer progression and to treat advanced disease.

Finding new effective treatments for melanoma has proved very challenging. High resistance to conventional chemotherapeutic agents and radiation is a hallmark melanoma. (Smalley and Eisen, 2003; Strauss et al., 2003). Research has now shifted to identifying melanoma gene mutations and the associated perturbations of signal transduction pathways, in the hope that more specific targeted therapies can be developed. Several cellular pathways important to cell proliferation, apoptosis and resistance or metastases have been shown to be activated in melanoma. Melanoma likely develops multiple defects, including loss of regulatory functions or the gain of anti-apoptotic or proliferative functions. Thus, therapeutic agents that could inhibit several signalling pathways simultaneously would be highly desirable. In addition, administration of standard chemotherapies in combination with new agents may afford the traditional chemotherapies a new lease of life in the melanoma context, if chemo resistance is inhibited. The challenge now is to develop selective agents to target these aberrant pathways.

The aberrant activation of the nuclear factor-kappa B (NFκB) pathway has been associated with melanoma growth, metastasis and escape from apoptosis. In vitro studies have demonstrated constitutively elevated NFκB binding in human melanoma cultures compared to normal melanocytes (Dhawan et al, 2004, McNulty et al, 2004). The expression of the NFκB subunit, RelA, has also been shown to be significantly elevated in human nevi and melanomas relative to normal skin. The NFκB pathway is therefore a potential target for treating melanoma. The constitutive activation of NFκB has several effects. Previous studies demonstrated the persistent activation of NFκB leads to increased chemokine production (e.g. CXCL8 and CXCL1) which stimulates angiogenesis thus promoting tumour formation in melanoma. In addition, activated NFκB is an important central regulator of a number of genes involved in antiapoptosis and proliferation (Mayo et al, 1997). In addition to its anti-apoptotic role, NFκB may also have a critical role in the development of chemoresistance in melanoma (Wang et al, 1999).

Activation of NFκB in melanoma has been shown to be due to constitutive activation of IKK, a key regulator of the NFκB pathway (Yang and Richmond, 2001). NFκB pathway inhibitors, and in particular IKK inhibitors, may provide a highly effective means of killing tumour cells by targeting them towards apoptosis.

A small molecule that selectively targets NFκB activation in melanoma without affecting NFκB's functions in normal cells therefore has significant therapeutic potential, as a single agent or in combination with existing chemotherapies.

One particular compound of interest is 1,1 dimethyl heptyl-(3S,4S)-7-hydroxy-Δ⁶-tetrahydrocannabinol (dexanabinol) which is disclosed in U.S. Pat. No. 4,876,276. In addition to being a non competitive NMDA receptor blocker, dexanabinol has been shown to inhibit NFκB (Juttler et al, 2004).

### SUMMARY OF THE INVENTION

We have now found a method of rapidly killing melanoma cells comprising contacting the cell with 1,1 dimethyl heptyl-(3S,4S)-7-hydroxy-. Δ⁶-tetrahydrocannabinol (INN dexanabinol), or a derivative thereof. The present invention contemplates melanoma cancer cells that are premalignant, malignant, metastatic or multidrug-resistant

Therefore, in accordance with a first aspect, the present invention provides dexanabinol, or a derivative thereof, for the treatment of melanoma.

The treatment of melanoma according to the invention may comprise inhibiting NFκB activity in a melanoma cancer cell by providing dexanabinol, or a derivative thereof, to the cell.

Alternatively, the treatment of melanoma may comprise the inhibition of tumourigenesis of a melanoma cancer cell by contacting the cell with an effective amount of dexanabinol, or a derivative thereof. Inhibition of tumourigenesis includes inducing both cytotoxicity and apoptosis in the cancer cell.

Furthermore, the treatment of melanoma according to the invention may comprise separately, simultaneously or sequentially inhibiting NFκB activity and tumourigenesis of a melanoma cancer cell.

Dexanabinol, or a derivative thereof, for the treatment of melanoma is advantageous, inter alia, because it shows reduced toxicity, reduced side effects and/or reduced resistance when compared to currently employed chemotherapeutic agents.

It is further contemplated that a second therapeutic agent may be provided in combination with dexanabinol, or a derivative thereof, to a melanoma cancer cell for treatment and/or prevention of melanoma. The second agent may comprise a chemotherapeutic agent, immunotherapeutic agent, gene therapy or radio therapeutic agent. The second therapeutic agent may be administered with the dexanabinol, or a derivative thereof, separately, simultaneously or sequentially.

The term “derivative” used herein shall include any conventionally known derivatives of dexanabinol, such as, inter alia, solvates. It may be convenient or desirable to prepare, purify, and/or handle a corresponding solvate of the compound described herein, which may be used in any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate. The term derivative shall especially include a salt. Suitable salts of dexanabinol are well known and are described in the prior art. Salts of organic and inorganic acids and bases that may be used to make pharmaceutically acceptable salts. Such acids include, without limitation, hydrofluoric, hydrochloric, hydrobromic, hydroiodic, sulphuric, nitric, phosphoric, citric, succinic, maleic, and palmitic acids. The bases include such compounds as sodium and ammonium hydroxides. Those skilled in the art are familiar with quaternizing agents that can be used to make pharmaceutically acceptable quaternary ammonium derivatives of dexanabinol. These include without limitation methyl and ethyl iodides and sulphates.

According to a further aspect of the invention we provide a method of treatment or alleviation of melanoma which comprises contacting a melanoma cell with a therapeutically effective amount of dexanabinol, or a derivative thereof.

Accordingly, and in one embodiment, the present invention provides a use of dexanabinol, or a derivative thereof, in the manufacture of a medicament for the treatment of melanoma.

We especially provide a use of dexanabinol, or a derivative thereof, in the manufacture of a topically administrable medicament, e.g. a topically administrable medicament for the treatment of melanoma.

Furthermore, in a second aspect, the present invention provides a method of treatment melanoma, said method comprising the administration of a therapeutically effective amount of dexanabinol and derivatives and/or combinations thereof.

Dexanabinol and derivatives and/or combinations thereof are known per se and may be prepared using methods known to the person skilled in the art or may be obtained commercially. In particular, dexanabinol and methods for its preparation are disclosed in U.S. Pat. No. 4,876,276.

Advantageously, in the use and or method of the invention the compound and derivatives and/or combination thereof may be administered orally, or intravenously.

Thus, in the use, method and/or composition of the invention of the compound may be put up as a tablet, capsule, dragee, suppository, suspension, solution, injection, e.g. intravenously, intramuscularly or intraperitoneally, implant, a topical, e.g. transdermal, preparation such as a gel, cream, ointment, aerosol or a polymer system, or an inhalation form, e.g. an aerosol or a powder formulation.

Compositions suitable for oral administration include tablets, capsules, dragees, liquid suspensions, solutions and syrups;

Compositions suitable for topical administration to the skin include creams, e.g. oil-in-water emulsions, water-in-oil emulsions, ointments, gels, lotions, unguents, emollients, colloidal dispersions, suspensions, emulsions, oils, sprays, foams, mousses, and the like. Compositions suitable for topical application may also include, for example, liposomal carriers made up of lipids or special detergents.

Examples of other adjuvants, diluents or carriers are:

for tablets and dragees—fillers, e.g. lactose, starch, microcrystalline cellulose, talc and stearic acid; lubricants/glidants, e.g. magnesium stearate and colloidal silicon dioxide; disintegrants, e.g. sodium starch glycolate and sodium carboxymethylcellulose;
for capsules—pregelatinised starch or lactose;
for oral or injectable solutions or enemas—water, glycols, alcohols, glycerine, vegetable oils;
for suppositories—natural or hardened oils or waxes.

It may be possible to administer the compound or derivatives and/or combination thereof or any combined regime as described above, transdermally via, for example, a transdermal delivery device or a suitable vehicle or, e.g. in an ointment base, which may be incorporated into a patch for controlled delivery. Such devices are advantageous, as they may allow a prolonged period of treatment relative to, for example, an oral or intravenous medicament.

Examples of transdermal delivery devices may include, for example, a patch, dressing, bandage or plaster adapted to release a compound or substance through the skin of a patient. A person of skill in the art would be familiar with the materials and techniques which may be used to transdermally deliver a compound or substance and exemplary transdermal delivery devices are provided by GB2185187, U.S. Pat. No. 3,249,109, U.S. Pat. No. 3,598,122, U.S. Pat. No. 4,144,317, U.S. Pat. No. 4,262,003 and U.S. Pat. No. 4,307,717.

For the treatment of melanoma, the invention especially allows administration of compositions topically on the skin or by injection, e.g. subcutaneously, or both. In additional the dexanabinol, or a derivative thereof, may be delivered simultaneously or sequentially by an injection method and topically to reduce the growth of a skin tumour.

Topically administered compositions are especially preferred.

The invention will now be illustrated by way of example only.

### DETAILED DESCRIPTION

### Example 1

### Induction of Apoptosis by Dexanabinol in Human Melanoma Cell Lines

### Methods

3 human melanoma cell lines (A375, G-361, WM266-4) were maintained in RPMI 1640 culture medium (Sigma, UK) containing 10% (v/v) heat inactivated foetal bovine serum (Sigma, UK) and 2 mM L-glutamate at 37° C. in 5% humidified CO₂. Cells were harvested, washed, re-suspended into growth medium and counted (Beckman-Coulter Vi-CELL XR). Cells were plated onto the middle 240 wells of 384 tissue culture plates at 1.6×10⁵ to 2.4×10⁵ cells/ml in 12.5 μl/well aliquots. 50 μl of growth media was aliquoted into the outer wells. 2 plates were prepared per cell line. Plates were incubated overnight at 37° C., in 5% humidified CO₂.

Dexanabinol was prepared in growth medium at 2 times the final assay concentration at 125, 31.3, 7.81, 2.00, 0.49, 0.12, 0.031 and 0.008 μM (DMSO concentration was kept constant across the dilution range at 0.5%).

Cisplatin was used as a positive control. The final assays concentrations were 10, 2.5, 0.63, 0.156, 0.039, 0.010, 0.002 and 0.0006 μg/ml. 12.5 μl per well of dexanabinol or cisplatin dilutions were added to the plates in replicates of 6. 12.5 μl of growth media was added to the media control wells. The plates were incubated for 24 hours at 37° C., in 5% humidified CO₂.

Caspase 3/7 levels were assessed by Apo-ONE© Homogeneous Caspase-3/7 assay kit. Fluorescence was measured using a FlexStation© II³⁸⁴ plate reader at 1, 2, 3 and 4 hours after addition of the caspase substrate. The 4 hour readings were used for analysis.

The cell viability assay was performed in parallel on the same plate for each line using CellTiter-Blue© (Promega) reagent. Briefly, 25 μl of CellTiter-Blue© (Promega) reagent was added to each well. The plates were shaken for 1 minute at 500 rpm and then incubated at 37° C., 5% CO₂ for 4 hours. Fluorescence was measured using a FlexStation© II³⁸⁴ plate reader (570 nm excitation wavelength, 600 nm emission wavelength, 590 nm cut-off.) The plots showing the cytotoxic effect of dexanabinol and cisplatin are shown as an overlay on the same graph.

### Results

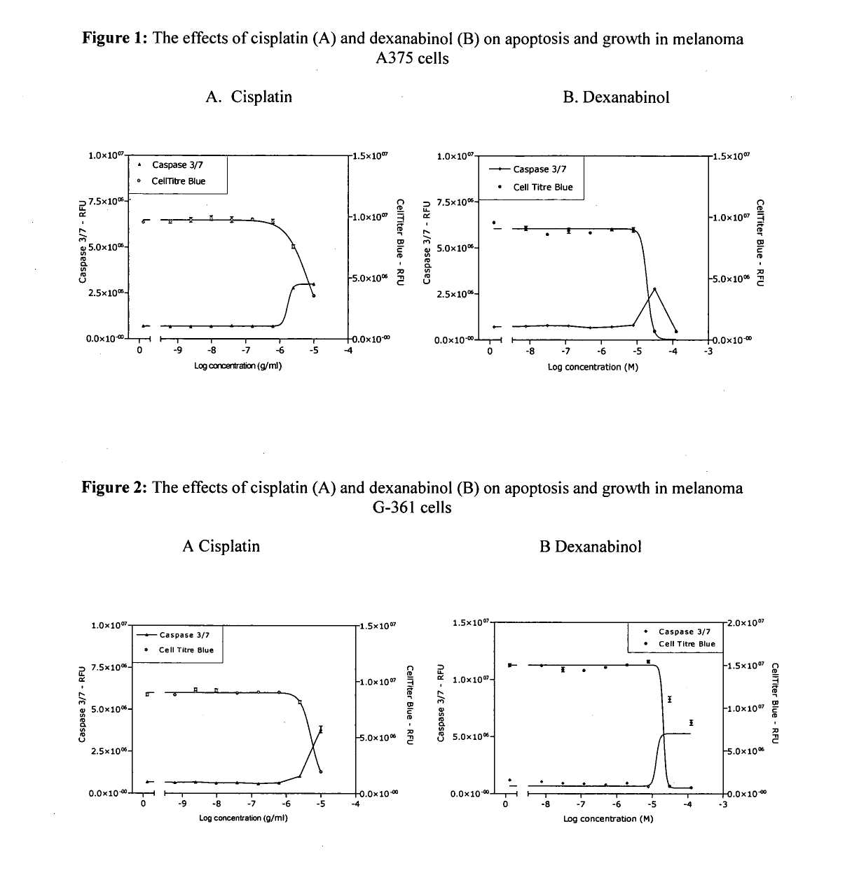

The induction of apoptosis in A375, G-361 and WM266-4 melanoma cell lines following 24 hours incubation with either cisplatin or dexanabinol is shown in FIGS. 1-3 respectively and summarized in Table 1. The assessment of cell viability as measured by the CellTiter-Blue® assay, indicating cytotoxicity is also shown.

Cisplatin was used as a positive control and a cytotoxic response was observed in all 3 melanoma cell lines with an approximate IC₅₀ value of 20-60 μM. The induction of apoptosis was not as easily quantified due to inadequate dose curves (G-361, WM266-4).

Dexanabinol induced a cytotoxic response with IC₅₀ values in the range of 10-21 μM in the 3 melanoma cell lines. The induction of apoptosis was not quantified in G-631 and A375 due to inadequate dose response curves. A peak response in apoptosis occurred at 2.5 μM and dropped at the highest concentration of 10 μM possibly due to cell lysis and loss.

TABLE 1
Effect of Dexanabinol on induction of apoptosis and
cell proliferation in 3 human melanoma cell lines.

| | Cisplatin | | Dexanabinol | |
|---|---|---|---|---|
| | ↑Apoptosis | ↓Viability | ↑Apoptosis | ↓Viability |
| Cell line | EC₅₀ (μM) | IC₅₀ (μM) | EC₅₀ (μM) | IC₅₀ (μM) |
| A375 | 5.67** | 21.8** | ND*** | 19.16** |
| G-361 | Approx 33.3-100** | 18.00** | ND* | 10.97*** |
| WM266-4 | Approx 33.3-100*** | 62.00* | 13.04*** | 20.87** |

ND - EC/IC₅₀ not determined due to inadequate dose response curve
Rank
*weak apoptosis induction and decrease in proliferation (<35%)
**moderate apoptosis induction and decrease in proliferation (35-70%)
***good apoptosis induction and decrease in proliferation (>70%)

The results are illustrated in FIGS. 1 to 3, in which;

FIG. 1 illustrates the effects of cisplatin (A) and dexanabinol (B) on apoptosis and growth in melanoma A375 cells;

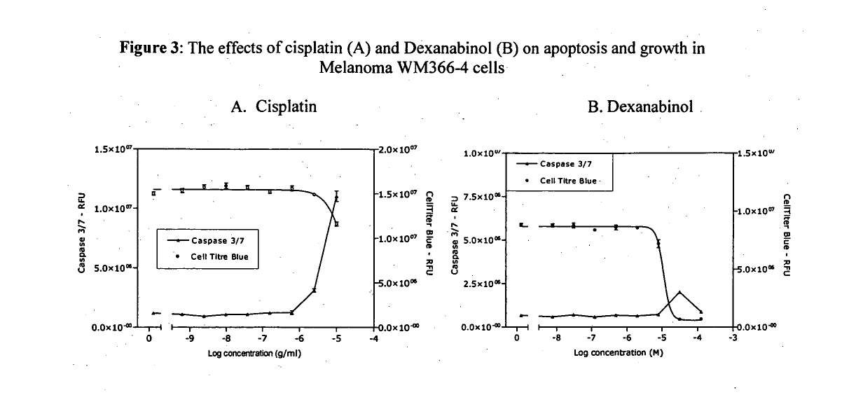

FIG. 2 illustrates the effects of cisplatin (A) and dexanabinol (B) on apoptosis and growth in melanoma G-361 cells; and

FIG. 3 illustrates the effects of cisplatin (A) and Dexanabinol (B) on apoptosis and growth in Melanoma WM366-4 cells.

### Example 2

### Inhibition of Melanoma Cell Proliferation by Dexanabinol

### Methods

The ability of dexanabinol to inhibit melanoma cell growth was also studied in 3 melanoma cell lines (A375, UACC62, Malme-3M) using the Sulforhodamine B (SRB) assay.

Sterile solutions of the final concentrations of dexanabinol were prepared (0.001 μM to 100 μM in 0.5% DMSO).

The cells were incubated with drug concentrations in 0.5% DMSO (100 μl total volume per well) at 37° C. in 5% CO₂ for 5 days. Control wells contained cells plus 0.5% DMSO plus medium.

The SRB growth inhibition assay was conducted over 24 and 5 day exposure periods. Following exposure, the cells were fixed, stained with SRB and read on a SpectorMax® 250 microplate spectrophotometer system plate reader.

### Results

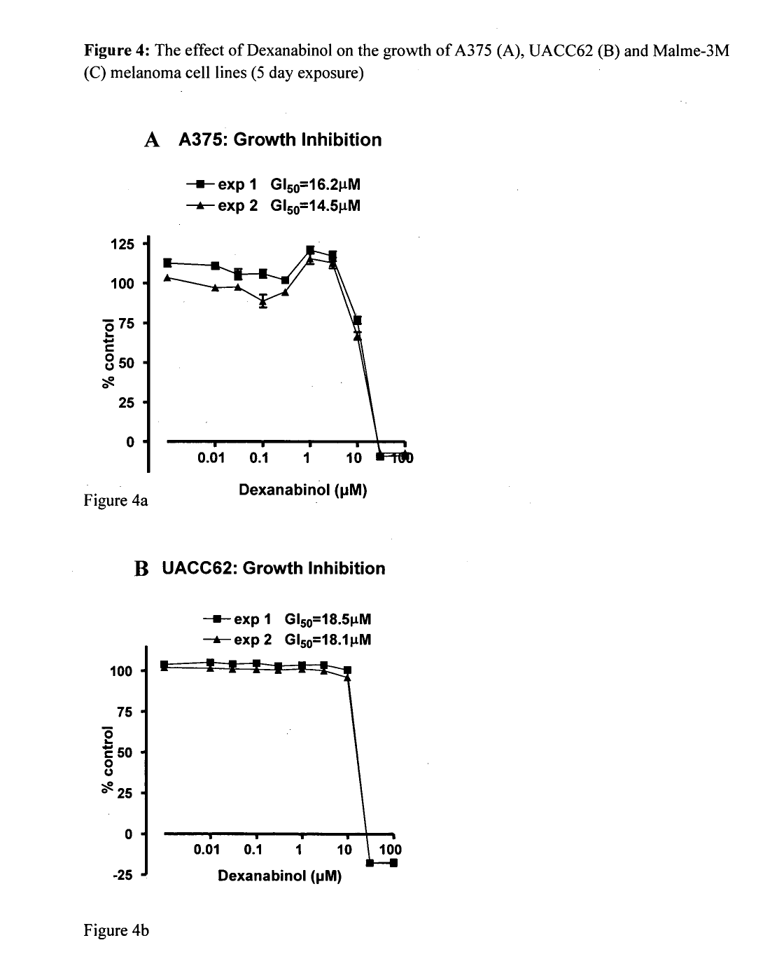

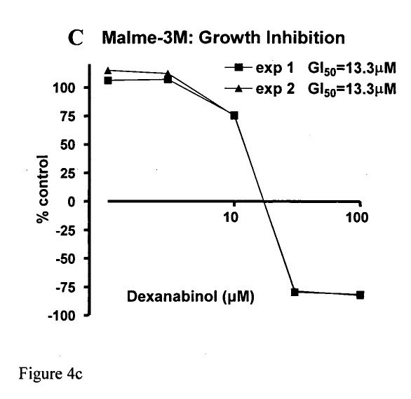

The effect of dexanabinol on the inhibition of growth in 3 melanoma cell lines after 5 days exposure is shown in FIG. 4. Growth inhibition was also measured after 24 hours exposure. GI₅₀ values for both exposure times are summarised in Table 2.

TABLE 2
Effect of exposure time on dexanabinol's ability
to inhibit melanoma cell growth in vitro.

| | | A375 | Malme-3M | UACC62 |
|---|---|---|---|---|
| GI₅₀ (μM)^{a} | 5-day | 16.2 | 13.3 | 18.5 |
| | | 14.4 | 13.3 | 18.1 |
| | 24-hour | 14.3 | | |

^{a}Measured by SRB growth inhibition assay

### Example 3

### Time-Course Effect of Dexanabinol on Cell Proliferation

### Methods

The time-course effect of dexanabinol on cell proliferation in one melanoma cell line, A375, was examined using the clonogenic assay method.

A375 cells were harvested from the growing cell culture and counted. Cells were diluted to 1.0×10⁵/ml and seeded 2 ml/well to 6 well plates.

The cells were incubated overnight at 37° C. in humidified incubator 95% air+5% CO₂.

The cells were treated with dexanabinol at 4 doses up to 20 μM at 3 exposure periods (1 hour, 6 hours and 24 hours).

After each exposure period, the cells were harvested from the wells by washing twice with PBS then adding 0.2 ml of single strength trypsin to each well and incubating at 37° C. until the cells detached. Cell suspensions were counted at 1/10 dilution and then diluted to 1/10, 1/100, 1/1000.

10 cm dishes (containing 7 ml fresh medium) were seeded with 3 different cell densities for dexanabinol treatment and control treatment. When colonies of suitable size formed in the control dishes, dishes were fixed, stained and counted.

Percentage survival in the dexanabinol treated A375 cells was calculated using the following equation:

$$Cloning efficiency \% = \frac{Colonies counted}{Cells seeded} \times 100$$

$$\% Survival = \frac{Drug treated cell cloning efficiency}{Control cell cloning efficiency} \times 100$$

### Results

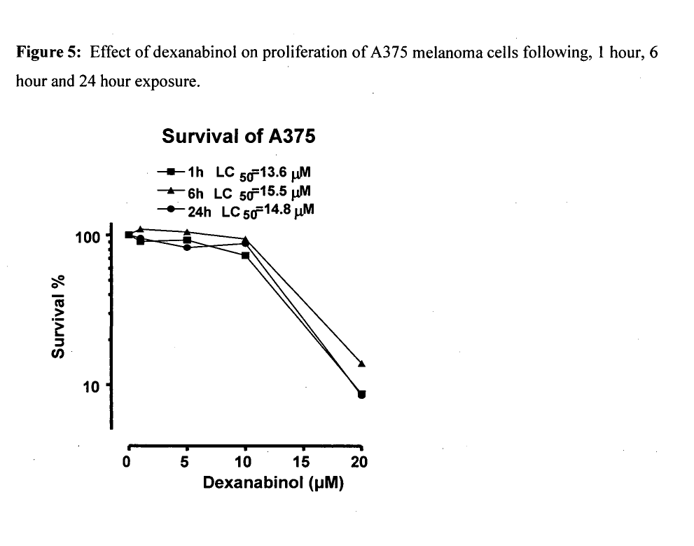

The time-course effect of dexanabinol on cell killing in A375 melanoma cells is shown in FIG. 5.

### Example 4

### Inhibition of Human Melanoma Cell Xenograft Growth by Dexanabinol

### Methods:

Having established that dexanabinol inhibited melanoma cell proliferation in vitro, we then sought to establish whether the compound was active in vivo. A preliminary pharmacokinetic (PK) and maximum tolerated dose (MTD) study was carried out to determine whether therapeutically effective dose levels of dexanabinol could be achieved in CD1 A375 tumour-bearing mice. The results demonstrated that at the MTD (100 mg/kg i.v. single dose) a plasma concentration of 10 μM was achievable for 2 hours. On the basis of this, a single-dose efficacy study was undertaken. Dexanabinol was diluted in a vehicle of 10% Cremophor/Ethanol (1:1 v/v) in saline. Control mice received vehicle alone.

10 mice received 100 mg/kg i.v. dexanabinol. 10 mice received Vehicle (10 ml/kg) Mice were implanted with 1×10⁷ A375 human melanoma cells, in 50 μl of medium, on the flank. Once tumours were palpable (approx 5 mm×5 mm) 20 mice were treated with either dexanabinol or vehicle (as outlined above). Mice were observed at least daily and weighed 3 times a week. Tumour size was measured 3 times a week after treatment. The treatment lasted 4 weeks

### Results

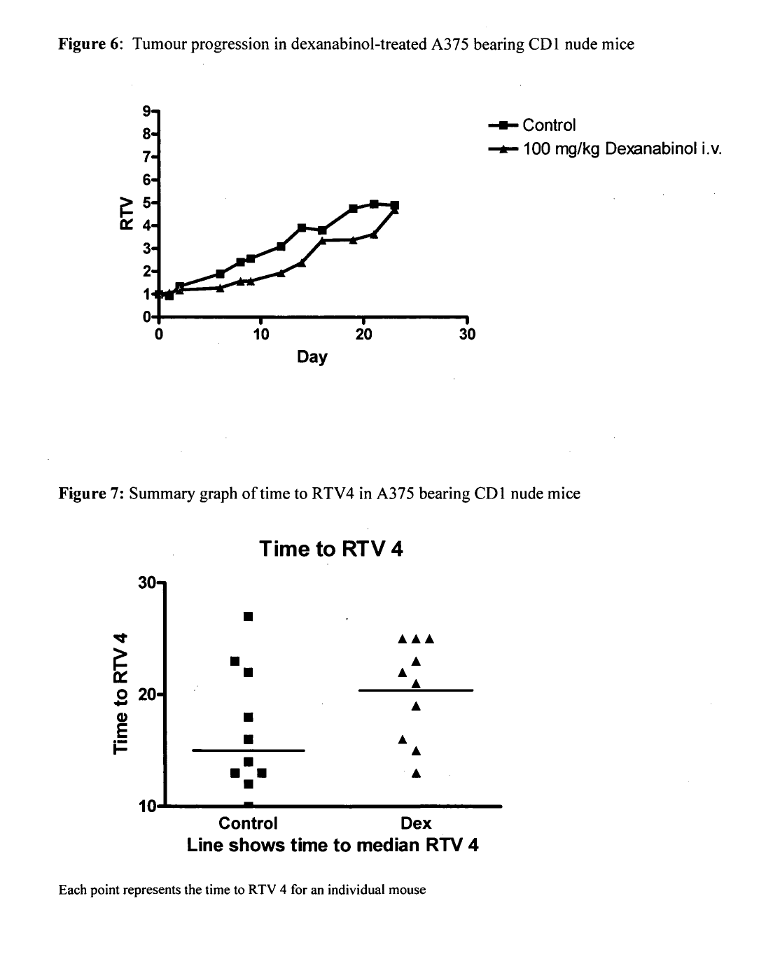

The time course of tumour growth and a summary graph of the time for tumours to reach a tumour volume 4 times that on the day of treatment (time to RTV 4) are shown in FIGS. 6 and 7.

### SUMMARY

Dexanabinol was tested against several established human melanoma cell lines, including some derived from disseminated melanoma metastases in other tissues. The in vitro cell proliferation assays showed dexanabinol to be profoundly cytotoxic to all tested human melanoma cell lines at concentrations distributed about a mean of 14 Dexanabinol induced apoptotic cell death in a caspase 3/7 dependent manner. This effect was time-independent, with cell killing being as profound after 1 hour as after 24 hours.

The in vitro anti-tumour effects of dexanabinol were observed at drug concentrations demonstrated to be safe and clinically achievable in patients (˜10-20 μM). The effect of a single dose of dexanabinol on human melanoma cell xenograft growth was then undertaken. The minimum required plasma concentration, based on the in vitro cell proliferation assay results, (10 μM maintained for at least 2 hours) was achieved by administering a single i.v. dose of dexanabinol at the MTD (100 mg/kg) in CD1 nude mice. The single dose of dexanabinol had a growth delaying effect in CD1 nude mice bearing the A375 human tumour xenograft.

1. Dexanabinol, or a derivative thereof, for the treatment of melanoma.

2. Dexanabinol according to claim 1 wherein the melanoma cancer cells are premalignant, malignant, metastatic or multidrug-resistant.

3. Dexanabinol according to claim 1 wherein the treatment of melanoma comprises separately, simultaneously or sequentially inhibiting NFκB activity in a melanoma cancer cell.

4. Dexanabinol according to claim 1 wherein the treatment of melanoma comprises inhibition of tumourigenesis of a melanoma cancer cell.

5. Dexanabinol according to claim 4 wherein the treatment of melanoma comprises the inhibition of tumourigenesis including inducing both cytotoxicity and apoptosis in the cancer cell.

6. Dexanabinol according to claim 1 in combination with a second therapeutic agent selected from one or more of a chemotherapeutic agent, an immunotherapeutic agent, a gene therapy or a radiotherapeutic agent.

7. A method of treatment or alleviation of melanoma which comprises contacting a melanoma cell with a therapeutically effective amount of dexanabinol, or a derivative thereof.

8. A method of treating a patient suffering from melanoma, said method comprising the administration of a therapeutically effective amount of dexanabinol, or a derivative thereof.

9. A method according to claim 8 wherein the melanoma cancer cells are premalignant, malignant, metastatic or multidrug-resistant.

10. A method according to claim 8 wherein the treatment of melanoma comprises separately, simultaneously or sequentially inhibiting NFκB activity in a melanoma cancer cell.

11. A method according to claim 8 wherein the treatment of melanoma comprises inhibition of tumourigenesis of a melanoma cancer cell.

12. A method according to claim 8 wherein the treatment of melanoma comprises inhibition of tumourigenesis of a melanoma cancer cell by inducing both cytotoxicity and apoptosis in the cancer cell.

13. A method according to claim 8 which comprises administration of a dexanabinol, or a salt or a derivative thereof, in combination with one or more of a therapeutic agent comprising a chemotherapeutic agent, an immunotherapeutic agent, a gene therapy, or a radiotherapeutic agent; separately, simultaneously or sequentially.

14. A method according to claim 8 wherein the therapeutically effective amount of dexanabinol, or a derivative thereof is administered orally or intravenously.

15. A pharmaceutical composition comprising dexanabinol, or a derivative thereof, and one or more of a therapeutic agent comprising a chemotherapeutic agent, an immunotherapeutic agent, a gene therapy, or a radiotherapeutic agent; in admixture with a pharmaceutically acceptable adjuvant, diluent or carrier.

16-20. (canceled)
